(19) United States
(12) Patent Application Publication
(10) Pub. No.: US 2024/0084368 A1
(43) Pub. Date: Mar. 14, 2024

(54) MICROFLUIDIC SYSTEM CONSISTING OF A FOLDED FOIL, AND PRODUCTION METHOD

(71) Applicant: Joanneum Research Forschungsgesellschaft mbH, Graz (AT)

(72) Inventors: Barbara Stadlober, Graz (AT); Jan Hesse, Linz (AT); Anja Haase, Graz (AT); Christian Wolf, Graz (AT)

(73) Assignee: Joanneum Research Forschungsgesellschaft mbH, Graz (AT)

(21) Appl. No.: 18/263,907

(22) PCT Filed: Feb. 17, 2022
(86) PCT No.: PCT/EP2022/053879

§ 371 Date: Aug. 2, 2023

(30) Foreign Application Priority Data
Feb. 18, 2021 (EP) 21157898.4

(51) Int. Cl. C12Q 1/6844; B01L 3/00; B01L 7/00

(52) U.S. Cl. CPC C12Q 1/6844; B01L 3/502707; B01L 3/502715; B01L 7/52; B01L 2300/0887; B01L 2300/123; B01L 2300/16; B01L 2300/1805

(57) ABSTRACT

A film composite folded from a single film to form a plurality of layered film layers (1) and comprising microfluidic structures (5) in different planes, wherein the microfluidic structures are formed by film layers having embossed recesses covered by the respective film layer adjacent in the layering direction, and wherein microfluidic structures of one plane are connected to microfluidic structures of another plane via through-holes in at least one film layer; a method for producing the film composite; and the use of the film composite.

### FIELD OF THE INVENTION

The present invention relates to a film composite comprising a folded film forming a microfluidic system, a method of manufacturing the film composite, and applications of the film composite.

### STATE OF THE ART

Components with microfluidic structures are typical elements in many sensor chips and contain microchannels and microchambers in which liquids can be absorbed, transferred, mixed or otherwise processed. Exemplary applications are biosensors for the rapid diagnosis of germs in hospitals or for the rapid analysis of chemicals in process technology, environmental analysis and the like. For most applications, the microfluidic structures are closed with a cover containing inlet and outlet ports.

A lab-on-chip system is a small mini-laboratory that is used, for example, for quick tests and analyses directly at the patient's home or at the family doctor's office. If such a system is integrated on a film substrate, it is called a lab-on-foil system. It should be small and easy to dispose of after use and therefore also inexpensive to produce. Lab-on-foil systems fulfil these conditions to a particularly high degree. Mostly, such systems are manufactured in the size of microscope slides in order to fit into conventional measuring set-ups. Due to the limited size of these systems, they often contain only a few functions.

DE 10 106 008 A1 discloses a microfluidic system as a PCR microreactor comprising a folded structure with two film layers having microfluidic structures and through-holes. The microfluidic structures are formed by forming recessions in one film layer, which are covered by folds through the second film layer having through-holes. Alternatively, the microfluidic structures are formed by forming recessions in both film layers that are mirror images of each other and folding the two film layers over each other to form microfluidic structures consisting of the recessions of the two film layers. Thus, a microfluidic system with microfluidic structures in different planes is not formed. In addition, the microfluidic system consists of only two film layers, so that no through-holes are included for connecting microfluidic structures of different planes.

US 2018/0264474 discloses a microfluidic system for tissue engineering comprising a folded structure with multiple film layers, wherein microfluidic structures in the film layers and through-holes through the film layers are created by laser.

### Problem to be Solved by the Invention

The prior art discloses microfluidic systems formed by film folding in which the microfluidic structures are not formed by embossing. Compared to laser processes, an embossing process has the advantages that it can be performed quickly and efficiently, especially when performed as a roll-to-roll process, and that microfluidic structures can be produced in a simple manner.

Therefore, it is the object of the present invention to provide an improved film composite with microfluidic structures using a simpler process.

### SUMMARY OF THE INVENTION

The problem was solved by providing a film composite which is formed by folding a single film and which has microfluidic structures produced by embossing.

More particularly, the object of the present invention is defined in the following points:
[1] A film composite which is folded from a single film to form a plurality of layered film layers (1) and comprises microfluidic structures (5) in different planes, wherein the microfluidic structures are formed in that film layers with embossed recesses are covered by the respective film layer adjacent in the layering direction, and wherein microfluidic structures of one plane are connected to microfluidic structures of another plane via through-holes in at least one film layer.

The microfluidic structures are thus formed by embossing recessions in one film layer and covering these recessions with another film layer. At least two, optionally more than two or all of these microfluidic structures in different planes are connected via through-holes, i.e. they are in communication with each other.
[1-1] Preferably, the microfluidic structures are formed without the use of an adhesive between the film layers.
[2] Film composite according to [1] or [1-1], which is folded in a fan-shaped manner or is obtainable by folding a film, which has two folding edges and thus a central film layer and a first and a second outer film layer, in such a way that the first outer film layer is folded over the central film layer and the second outer film layer is subsequently folded over the first film layer.

According to [2], the folding is carried out in a fan-shaped manner, i.e. according to the fanfold principle. According to [2], the folding is alternatively carried out according to the principle shown in FIGS. 5 and 6. The advantage of this principle is that, in the case of a film with three film layers, the two folding edges can be formed on the same side of the film and all the recessions can be embossed on the same side of the film and, after folding, all the recessions are still on the inside of the film composite, i.e. no recession is on the outside.
[3] The film composite according to [1], [1-1] or [2], wherein (i) at least one of the film layers comprises a carrier film and, on one surface side of the carrier film, a lacquer layer with embossed recesses; and/or wherein (ii) at least one of the film layers comprises a carrier film and, on both surface sides of the carrier film, a lacquer layer with embossed recesses on at least one of the two surface sides of the carrier film.

In the case of (ii), microfluidic structures may be formed in different planes by (a) covering recessions in two adjacent film layers and/or (b) having recessions on both surface sides of a film layer and covering the recessions on both surface sides.

When the film is folded in a fan-like manner, a film composite according to item [3](i) may be preferred which is folded from a film having alternately a section with embossed lacquer layer on one surface side and a section with embossed lacquer layer on the other surface side and which is folded between the sections to form the film layers. In this way, a carrier film can be prevented from being covered by a carrier film in the entire film composite.

Film composite according to point [3](ii) may be preferred, with recesses on both surface sides of the carrier film. In this case, microfluidic structures of different planes are created during each folding process without an intermediate carrier film.

In the case of two adjacent film layers, the surface sides that have the recesses may face each other. The facing recessions can at least partially overlap to form a higher microfluidic structure that can be used, for example, as a reaction chamber.
[3-1] Preferably, in the film composite according to [3] the lacquer layers of two adjacent film layers are bonded by UV curing.

UV curing forms covalent bonds, resulting in a particularly strong connection of the layers and a hermetic covering of the microfluidic structures.
[4] The film composite according to any of the preceding points, wherein the film is folded at folding edges created by laser cutting of the films and/or wherein the through-holes are created by laser cutting.
[5] The film composite according to any one of the above points, which consists of congruent film layers, which lie directly on top of each other over their entire surface, and folded edges.
[5-1] Preferably, the film composite according to [5] is folded in a fan-like manner.
[5-2] Preferably, in the film composite according to [5], the lacquer layers of two adjacent film layers are bonded by UV curing.
[6] The film composite according to any one of the preceding points, wherein microfluidic structures communicating with one another are formed in different planes in that two film layers adjacent in the layering direction have embossed recessions which face one another and only partially overlap one another in the layering direction.

The facing recessions overlap each other, but only partially. They are therefore not congruent after folding. This means that when the two layers of film are folded on top of each other, microfluidic structures are created that lie in different planes and are connected in a communicating manner, whereby no through-hole is required, which would have to be produced with the help of a laser, for example.
[7] A film composite according to any one of the preceding points, comprising in the microfluidic structures at least one modification selected from functionalisation, nanostructuring, hydrophobisation and heating agent filling.

A microfluidic structure may have several of these modifications, with the modifications being present in different sections of the microfluidic structure. By placing two recessions on top of each other during folding, two separate and different modifications can even be present in the same place in the microfluidic structure in plan view, namely one modification at the top and one at the bottom.
[8] The film composite according to any one of the preceding points, wherein at least one of the film layers has a carrier film and, on at least one surface side of the carrier film, a lacquer layer with embossed recessions, wherein either (i) the recessions are present in a lacquer layer of the film layers and the lacquer layer is bonded to the adjacent film layer by UV curing or (ii) the recessions are present in a thermoplastic lacquer layer of the film layers and the lacquer layer is bonded to the adjacent film layer by exclusively thermal bonding.

The film composite according to item [8](i) is described in the first embodiment. The film composite according to item [8](ii) is described in the second embodiment
[9] The film composite according to [1], which is folded in a fan-shaped manner and consists of congruent film layers, which lie directly over one another over their entire surface, and folding edges, wherein the folding edges and the through-holes have been produced using a laser and wherein the film layers have a carrier film and a lacquer layer with recessions and the lacquer layer is bonded to the adjacent film layer by UV curing.
[10] A method of producing a film composite according to any one of [1] to [9], comprising the steps of
   (a) applying a layer of a curable embossing lacquer to a first surface side of a carrier film to form a embossing lacquer layer in an embossing lacquer section,
   (b) applying a layer of a curable bonding lacquer to the first surface side of the carrier film to form a bonding lacquer layer in a bonding lacquer section,
   (c) embossing recessions in the embossing lacquer layer obtained in step (a) with an embossing tool,
   (d) partial curing of the embossing lacquer layer embossed in step (c),
   (e) removing the embossing tool after step (d),
   (f) forming through-holes through the structure obtained in step (e) from the first surface side to the second surface side of the carrier film and folding the structure between the embossing lacquer section and the bonding lacquer section to form two film layers,
   (g) superimposing the two film layers obtained in step (f) to form a composite so that the recessions of the embossing lacquer layer of the embossing lacquer section of one film layer are sealed by the bonding lacquer layer of the bonding lacquer section of the other film layer to form microfluidic structures,
   (h) curing the partially cured embossing lacquer layer and the bonding lacquer layer of the composite obtained in step (g) to form covalent bonds between these layers, thereby obtaining the film composite.
[11] The method according to [10], wherein the curable bonding lacquer is a curable embossing lacquer which is embossed and partially cured according to steps (c) to (e).
[12] A method of producing a film composite according to any one of [1] to [9], comprising the steps of:
   (a) applying a layer of an embossing lacquer containing monomers radically polymerisable to a polymer to a first surface side of a carrier film to form an embossing lacquer layer in an embossing lacquer section,
   (b) applying a cover layer to the first surface side of the carrier film to form a cover layer in a cover layer section,
   (c) embossing recessions in the embossing lacquer layer obtained in step (a) with an embossing tool,
   (d) polymerising the monomers contained in the embossing lacquer layer into a polymer to form a thermoplastic embossing lacquer layer on the carrier film,
   (e) removing the embossing tool after step (d),
   (f) forming through-holes through the structure obtained in step (e) from the first surface side to the second surface side of the carrier film and folding the structure between the embossing lacquer section and the cover layer section to form two film layers,
   (g) thermally bonding the two film layers obtained in step (f) to each other so that the recessions of the embossing lacquer layer of the embossing lacquer section of one film layer are sealed by the cover layer of the cover layer section of the other film layer to form microfluidic structures, whereby the film composite is obtained.
[13] The method according to [12], wherein the cover layer is a thermoplastic embossed lacquer layer which is embossed and polymerised according to steps (c) to (e).
[14] The method according to any one of points [10] to [13], wherein the embossing is carried out in a roll-to-roll process.
[15] The use of a film composite according to any one of points [1] to [10] for the amplification of DNA by polymerase chain reaction or isothermal amplification.

### Advantages of the Invention

By using thin film layers with microfluidic structures, a larger available space is obtained for the same footprint and more functions can be accommodated in the same area.

The production of the film composite according to the invention is facilitated by folding the film layers at predetermined folding lines.

Roll-to-roll embossing of the microstructures over a large area and in high throughput can be used to produce the microfluidic structures in the film composite according to the invention. These microstructures can be simultaneously sealed by lamination of a cover layer using known methods. This makes it possible to create closed channel networks, which are necessary for use in biosensors, for example.

The microfluidic structures are tightly and hermetically sealed all around between the cover and the side walls. This makes the microfluidic structures mechanically stable and insensitive to pressure differences and stresses during handling. A hermetic seal also prevents the ingress of foreign substances and impurities that could falsify the analysis result, and also allows carrying out temperature-controlled processes (e.g. on-chip amplification). At the same time, the structure of carrier films and thin lacquer layers results in a high degree of mechanical flexibility, which enables production in cost-effective roll processes and easy folding. The microfluidic structures are also sealed without the use of an adhesive, thus avoiding possible contamination.

Since the microfluidic structures can be made of the same material all around, uniform and trouble-free transport of the fluids or analyte is possible.

The method according to the invention enables simple UV bonding or thermal bonding and can thus be used in high-throughput processes. In UV bonding, covalent bonds are generated. In addition, UV bonding does not require thermoplastic behaviour and thus does not restrict the use of monofunctional acrylates, thus allowing more freedom in material development. Due to the formation of cross-links during curing, the channel materials are elastic, so that cracking is avoided during roll-based production or in the application.

The multitude of communicating microfluidic structures of different sizes, shapes and properties enables a combination of different functions on a single substrate, thus saving time, costs and resources. For example, such a combination includes one or more of the following functions: sample amplification (e.g. by means of LAMP (Loop-mediated isothermation). LAMP (Loop-mediated Isothermal Amplification) or PCR (Polymerase Chain Reaction)) so that the DNA to be analysed can be amplified directly in the chip; the formation of hydrophobic retardation structures so that the fluids flowing through the chip can be stopped at desired positions; the formation of structured sensor surfaces to create a larger surface area and thus enable improved biofunctionalisation; an enlarged reaction chamber; an enlarged storage chamber to accommodate additional process fluids; the design of through-holes between the film layers in such a way that they draw a fluid from layer to layer in a capillary-driven manner; and the creation of heating conductor structures for the temperature control required during sample amplification, which are carried out by capillary-driven filling of the microfluidic structures with metallic inks and subsequent sintering (heating agent filling).

By forming air-filled chambers, the thermal insulation of chambers tempered by means of film heating can be improved. Retroreflectors can also be built into such air-filled chambers, which can better conduct the optical signal (for example of a chemiluminescence reaction) to the outside.

### DESCRIPTION OF THE FIGURES

FIG. 1 illustrates the principle of the production of the film composite according to the invention using the example of a film composite for use in the amplification of DNA. The temperature for the isothermal PCR is set via the heating medium-filled microfluidic structures (15) under the PCR chamber (13). The microstructured surface (14) may have biofunctionalisation (e.g. binding of DNA and/or protein). The hydrophobic valve (12) is used for selective interaction of the surface with substances and, for example, selective passage to adjacent microfluidic structures.

FIG. 2A shows a schematic cross-section of a film layer (1). An embossing lacquer layer (3) with indentations (4) of a depth of 100% is applied to one side of the carrier film (2).

FIG. 2B shows a cross-section of a film composite according to the invention consisting of film layers which have an embossed lacquer layer on one side (cf. FIG. 2A). The folded edge is not shown. The recessions are covered by the adjacent film layers to form microfluidic structures (5) which are closed in cross-section. The through-holes (6) connect microfluidic structures (5) of different planes.

FIG. 3A shows a schematic cross-section of a film layer (1). An embossing lacquer layer (3) with recessions (4) of a depth of 100% is applied to both sides of the carrier film (2). The recessions (4) can have different functionalities. For example, a recess (4) can contain heating means (7) or nanostructures (8).

FIG. 3B shows a cross-section of a film composite according to the invention consisting of film layers which have an embossed lacquer layer on both sides (cf. FIG. 3A). The folded edge is not shown. The recessions are covered by the adjacent film layers to form microfluidic structures (5) which are closed in cross-section. The through-holes (6) connect microfluidic structures (5) of different planes. The microfluidic structures (5) can have different functionalities. For example, a microfluidic structure (5) may contain heating means (9) or nanostructures (10).

FIG. 4 shows a cross-section of a film composite according to the invention consisting of film layers which have an embossed lacquer layer on both sides. The folded edge and the through-holes are not shown. The recessions are covered by the adjacent film layers to form microfluidic structures (5) which are closed in cross-section. A microfluidic structure (5) may have different functionalities in cross-section. For example, a microfluidic structure (5) may contain heating means (9) and nanostructures (10). A microfluidic structure (5) can form a larger chamber (16) by forming wide recessions (4) (cf. FIGS. 2A, 2B and 3A) of a depth of 100%, which can be used as a reaction chamber or storage chamber. The chamber (16) is connected to a microfluidic structure (5) via a connecting recess (17).

FIG. 5 shows a top view of a film for the production of a film composite according to the invention. The film consists of three film layers A, B and C and two folded edges (11). The film layers have through-holes (6) and recesses (4) on the upper side. A first folding (marked “1.” in FIG. 5) of film layer (A) onto film layer (B) creates a composite (A)/(B). A second folding (marked “2.” in FIG. 5) of film layer (C) onto film layer (A) of the composite (A)/(B) creates the film composite (B)/(A)/(C). The recess in (A), together with the through hole in (A) and the corresponding through hole in (B), results in a liquid inlet. The chamber in (B) can serve as a reaction chamber, e.g. for LAMP. Due to the non-conforming recessions in (A) and (B), the folding of (A) and (B) creates two chambers that lie in different planes and are communicatingly connected, whereby no passage opening is required. The recessions formed in (C) result in chambers when folded, which can serve, for example, as a sensor channel and waste reservoir, whereby the sensor channel is communicatingly connected to the reaction chamber in (B) via a through-hole in (A).

FIG. 6 is a sectional view of the film composite formed by folding the film shown in FIG. 5.

In the Figures, the microfluidic structures have a rectangular shape and mostly a depth of 100% of the layer thickness of the embossing lacquer layer. It should be noted that the microfluidic structures can have any other shape that can be produced by embossing, for example a triangular shape tapering towards the carrier film or rounded shape.

Furthermore, the depth can also be less than 100%, so that the microfluidic structure is not partially covered by the carrier film.

### EMBODIMENTS OF THE INVENTION

The film composite according to the invention is folded from a single film. That is, the film composite consists of a film that is folded. Consequently, other components, for example adhesives, or other layers or plies between the film layers are excluded, whereas additional components, such as nanostructures, heating agents, functionalisations and the like, are not excluded in the microfluidic structures. The film is folded several times and forms several film layers and folded edges in between.

In the film composite, microfluidic structures are contained in different layers, wherein (i) the microfluidic structures are formed in that film layers with embossed recessions are covered by the respective film layer adjacent in the layering direction, and wherein (ii) microfluidic structures of one layer are connected to microfluidic structures of another layer via through-holes in at least one film layer. Feature (ii) defines microstructures, i.e. recessions, on both sides of a film layer. These microstructures are created according to feature (i) by the fact that the recessions are each covered by a further film layer. The combination of features (i) and (ii) thus requires that the film composite has at least three film layers.

In one embodiment, the film composite is designed in such a way that the layered film plies lie on top of each other over their entire surface, i.e. that they are also connected immediately next to a folded edge. The film composite thus consists of congruent film layers, which lie directly on top of each other over their entire surface, and folded edges. The film layers have the same length and width and surface shape, preferably they are rectangles of the same size, and the folding edges are located in the vertical direction of the film composite alternately at one end and at the opposite end of the film layers, preferably on the opposite surface sides of the rectangular film layers. The recessions are not congruent or also congruent independently of the congruent film layers.

The film composite can thus preferably be folded according to the fanfold principle, i.e. fan-like, such as a stack of continuous paper. This means that there are no areas on at least one surface side of a film layer that are not connected to an adjacent film layer. In this way, the surface of the film layers is used to the maximum.

Alternatively or in addition to folding according to the fanfold principle, any type of folding is of course possible. For example, a film that has two folding edges and thus a middle film layer as well as a first and a second outer film layer can be folded in such a way that the first outer film layer is folded over the middle film layer and the second outer film layer is then folded over the first film layer.

As used herein, in the context of film layers, the term “layered film layers” means that each film layer is directly connected to another film layer in the film composite. As used herein, the term “layering direction” means the z-direction perpendicular to the x-y-plane of a flat film layer and indicates a position above or below the film layer. In the context of film layers, the expression “adjacent in the layering direction” means that a first film layer is directly connected to a second film layer and is above or below the second film layer in the z-direction. Herein, the expression “in different planes” means different height in the z-direction. A different plane of, for example, microfluidic structures can result from the fact that the microfluidic structures within a film composite are present in different film layers or on the lower and upper side of the same film layer.

In the film composite, at least some of the microfluidic structures of different planes are interconnected via through-holes. In this context, the term “interconnected” means that the microfluidic structures can communicate with each other, i.e. that it is possible to move materials, e.g. fluids such as liquids, from one microfluidic structure to another microfluidic structure.

A film layer is defined by the fact that it is separated from another film layer by folding the film, in particular by a folded edge.

The film and thus also each film layer is flat with dimensions in the x- and y-direction, which define the contact area with the film layers adjacent in the layering direction, and the z-direction, which corresponds to the film thickness or film layer thickness. The extents in x- and y-direction are each at least ten times, preferably at least twenty times, more preferably at least fifty times the extent in z-direction. The film layer is a thin film in itself.

Whenever the singular is used in the present invention to designate elements or structures, for example “a microfluidic structure” or “the microfluidic structure”, this is not intended to expressly exclude a plurality of these elements or structures, so that the exemplary designations are synonymous with “at least one microfluidic structure” or “the at least one microfluidic structure”, unless otherwise indicated.

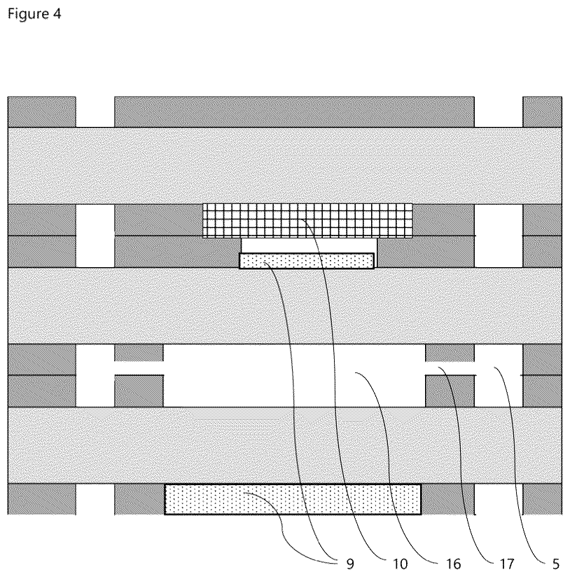

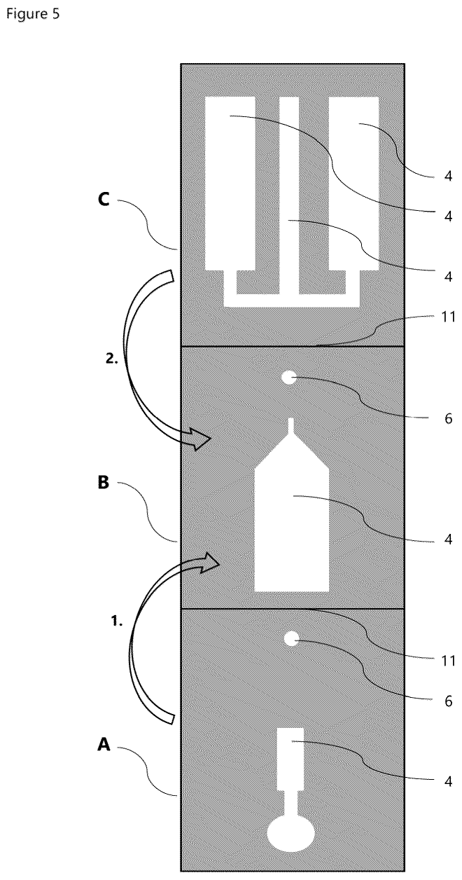

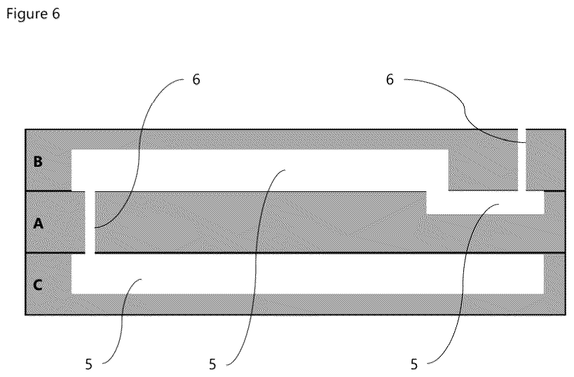

Heating may be required in the film composite for certain applications. This can be provided in a number of ways. It can be formed by filling a microfluidic structure with a heating medium and can thus be an integral part of the film composite, such as heater (9) in FIG. 4. However, the heater can also be attached to the outside of the film composite. The heating agent can be filled into embossed recesses of an outer film layer. For example, a metal ink can be filled in as the heating agent by means of capillary action. In this way, microfluidically filled electrodes can be created, which can be used as film heating. The heating can also be provided by applying a separately manufactured heating film. The heating agent can also be printed on an external film layer, for example by inkjet printing or screen printing. FIG. 6 shows a film composite suitable for one of the aforementioned heaters. In particular, the heater could advantageously be formed on the outside in or on film layer (B), i.e. on the back of the side having the recess. Both a film heater and a printed heater would be suitable for the film composite shown in FIG. 6.

The film consists of a carrier film and optionally on one or both of its surface sides further layers, for example an embossing lacquer layer or bonding layer.

The film layers for the film composite are folded from the film.

### Carrier Film

The carrier film is a flat polymer film with an area in the x-y direction and a thickness in the z direction.

The carrier film preferably has a thickness of 5 to 2000 μm, preferably 10 to 1000 μm, particularly preferably 20 to 500 μm. It is preferably a plastic film made of PI, PP, MOPP, PE, PPS, PEEK, PEK, PEI, PSU, PAEK, LCP, PEN, PBT, PET, PA, PC, COC (cycloolefin copolymer), POM, ABS, PVC, PTFE, ETFE (ethylene tetrafluoroethylene), PTFE (polytetrafluoroethylene), PVF (polyvinyl fluoride), PVDF (polyvinylidene fluoride), and EFEP (ethylene-tetrafluoroethylene-hexafluoropropylene-fluoropolymer), COP (cycloolefin polymer), PS (polystyrene).

The surface of the carrier film can have functionalisations, for example hydroxy, thio or amine groups. Alternatively, the functionalisations may be protected with protective groups, which are removed after the embossing and polymerisation of the base material of the embossing lacquer layer. The protection of the functionalisations can also amount to hydrophobisation of the surface, so that the materials of the carrier film and the embossing lacquer layer become more compatible and thus the non-covalent adhesion is strengthened.

The functionalisations can serve for covalent bonding of the polymers of the embossing lacquer layer.

The functionalisations can also serve to covalently bind desired compounds for diagnostic purposes, for example. Such compounds can be biomolecules, for example nucleic acids, proteins such as antibodies, lipids or carbohydrates. In addition, molecules with two reactive groups can be attached via these functionalisations, whereby one reactive group forms a covalent bond with the functionalisation of the carrier film and the other reactive group can react with a target substance.

As a rule, however, possible functionalisations are in or on the embossing lacquer.

### Embossed Lacquer Layer

The lacquer layer that is present on the carrier film and has the embossed recessions is referred to as the embossing lacquer layer. It has a thickness, i.e. an extent perpendicular to the contact surface with the surface side of the carrier film, of preferably 10 nm to 1000 μm, more preferably 50 nm to 500 μm, even more preferably 100 nm to 100 μm. In individual embodiments, the thickness may be 100 nm to 50 μm.

The depth of the embossing depends on the thickness of the lacquer layer and the depth of the stamp structures. The embossing depth can be 1 to less than 100% or even 1 to 100% of the thickness of the embossing lacquer layer. Preferably it is 50 to 100%, more preferably 50 to less than 100%, even more preferably 80 to 95% of the thickness of the embossing lacquer layer. The combination of the thickness of the embossing lacquer layer (in μm) and the embossing depth (in %) may preferably be 0.1 to 500 μm and 10 to 100%, more preferably 0.1 to 100 μm and 70 to 100% or 0.1 to 50 μm and 30 to 100%. In individual embodiments, the embossing depth may be 50 to 200 μm and may be 50 to 100% of the thickness of the embossing lacquer layer.

Typically, the embossing depth is less than 100% of the thickness of the embossing lacquer layer, so that the microfluidic structure has the same material on three sides (two walls and bottom). If the cover of the microfluidic structure is made of the same material as the embossing lacquer layer, the microfluidic structure is formed by the same material all around.

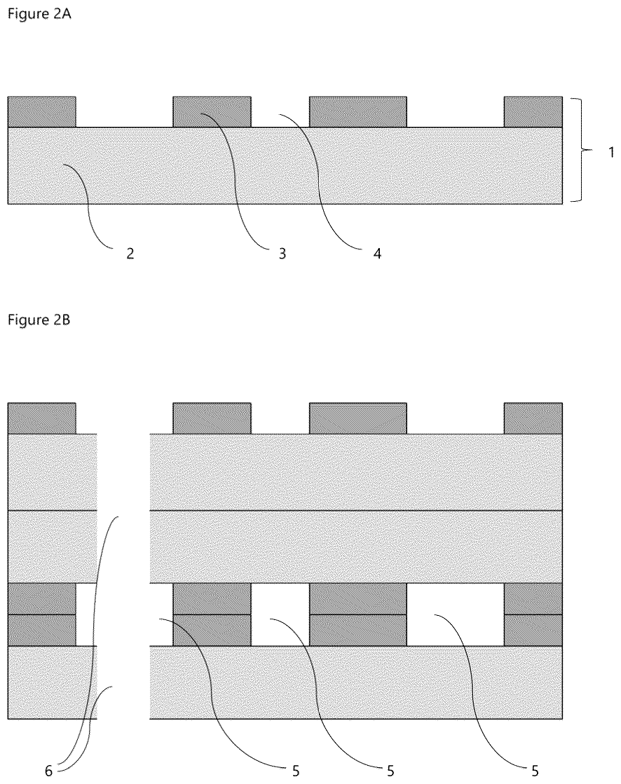

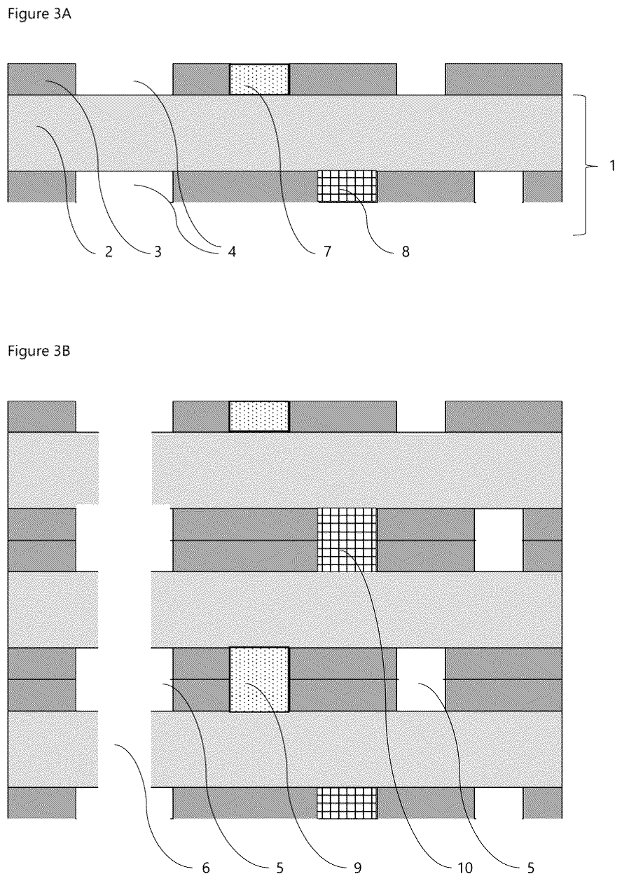

In a particular embodiment, the embossing depth may be 100% of the thickness of the embossing lacquer layer, meaning that there is an area within the embossing where no lacquer layer material remains on the carrier. In other words, the resist layer may have recessions that extend to the carrier film so that the microfluidic structure formed is at least partially covered by the carrier film. In this case, the embossing lacquer layer is not a continuous layer but an interrupted layer (cf. FIGS. 2 to 4). In general, these interruptions of the embossing lacquer layer at the contact surface to the carrier film preferably make up 1 to 90%, more preferably 5 to 70%, even more preferably 10 to 60% of the total area of the embossing lacquer layer. If the embossing depth is 100% of the thickness of the embossing lacquer layer, there is a surface of the carrier film in the embossed area which is free of embossing lacquer layer, i.e. accessible, and which may differ from the surface of the recessions in the embossing lacquer layer. These differences can consist, for example, in the fact that the surface of the carrier film has functionalisations, for example hydroxyl, thio or amine groups. At an embossing depth of 100%, the cavities extend from the underside of the cover layer to the surface of the carrier film, with thickness regions of the embossing lacquer layer forming the side walls of the cavities. The functionalisations on the surface of the carrier film exposed in the embossed region of the embossing lacquer layer can be used to bind desired compounds, preferably covalently, to the carrier film, as described further above. In this way, compounds can be selectively introduced into the embossed areas of the embossing lacquer layer without the need for a bonding option to the embossing lacquer layer. This gives greater freedom in the selection of the base materials of the embossing lacquer layer.

### Microfluidic Structures

The film composite according to the invention contains microfluidic structures, which are voids created by covering a film layer that has indentations with an adjacent film layer. They are thus sandwiched between two films.

The microfluidic structures can be formed from recessions in an embossed lacquer layer on a carrier film, which are covered by the adjacent film layer, i.e. a cover layer. The embossing lacquer layer is embossed on the surface side facing the cover layer. The cover layer is directly on top of the lacquer layer. More preferably, the embossed side of the lacquer layer, with the exception of the recessions of the embossing, is fully and directly covered by the cover layer. Preferably, there is neither lacquer layer material nor cover layer material in the recessions of the embossing, so that cavities are created which can be fluid-filled. The term “directly” means that no further material, for example an adhesive, is present between the lacquer layer and the cover layer.

The microfluidic structure has dimensions on a micrometre scale in the z-direction and either in the x-direction or y-direction, the x-y plane being defined by the areas of the film layers. For example, the microfluidic structure has a depth (in the z-direction) and width (x- or y-direction) of 0.1 to 200 μm each, preferably 1 to 50 μm. The length of the microfluidic structure is not limited. The microfluidic structure is suitable for receiving and transporting fluids, i.e. gases or liquids. A fluid at 21° C. is a liquid or a gas, in particular air. The microfluidic structure may also be filled with a solid material that is different from both the material of the embossing lacquer layer and the material of the cover layer. Such a material is, for example, a heating agent.

At 100% embossing depth, the cavities are formed between the carrier film and the adjacent film layer, at a lower embossing depth between the embossing lacquer layer and the adjacent film layer.

A microfluidic structure is tightly sealed all around. The term “all around” in reference to the cover means here that a microfluidic structure is sealed at every point in the z-direction, i.e. in the direction perpendicular to the substrate film plane, and in the x-direction or y-direction. Thus, with the exception of regions with through-holes or interconnect recesses, there is a cross-sectional plane in the y-direction at each point of a microfluidic structure in which the microfluidic structure is terminated all around. The microfluidic structures have at least one opening, preferably at least one inlet opening and at least one outlet opening, preferably at one of their ends in the x-y plane.

### Embossing

The film layers have embossed recessions. As used herein, “embossing” refers to a process in which a sufficiently soft substrate is contacted with an embossing tool having the negative shape of the desired indentation such that the desired indentation is created in the substrate.

The substrate can be an unpolymerised or incompletely polymerised or cured polymer starting material that is polymerised or cured after embossing. However, the substrate may also be a softened polymer that is solidified after embossing. For example, the solid polymer may be thermally softened, embossed and then re-solidified by cooling. Similarly, the solid polymer may be thermally melted in an extruder and re-solidified by cooling after extrusion and embossing. The embossing tool, which has the negative shape of the desired recession, can be, for example, an embossing die or a roller.

One example of an embossing process is ultraviolet nanoimprint lithography (UV-NIL). In this process, a liquid embossing lacquer is applied to a polymer substrate in a cold state. A nanostructured stamp with the inverted profile of the recessions is used as a negative mould and pressed into the embossing lacquer, in which the desired structure is then embossed as a positive mould. The embossing lacquer is polymerised and solidified by irradiation with UV light. After separating the stamp from the embossed pattern, the profile of the stamp is replicated in inverted form. This process can be implemented in a continuous roll-to-roll process when using a cylindrical stamp. This allows very large areas to be structured in a short time.

Examples of embossing operations are explained below in the description of the film composite of the first and second embodiments.

Another example of the formation of embossed recessions in a film composite according to the invention is the extrusion of a polymer and embossing with a roller.

In any case, during embossing there is contact and thus interactions between the sufficiently soft substrate and the solid embossing tool. These interactions are, for example, hydrophobic or ionic interactions. Since the substrate is not rigid, the molecules it contains are mobile. Due to the interactions with the embossing tool, there is in any case a certain orientation of the substrate molecules and thus a change in the substrate surface, which can be detected on the embossed and solid substrate. For example, the embossed surfaces differ from the non-embossed surfaces of the substrate in their wettability with water.

This change in the structure and thus also in the properties of the embossed substrate surface also makes it possible to distinguish an embossed indentation from an indentation that was created with the help of a laser, for example. The substrate of a laser treatment is not soft, but rigid, so that an orientation of the substrate molecules and thus a change of the surface properties is excluded. Furthermore, in contrast to an embossing process, laser treatment, due to its nature as a subtractive process and due to the high energy input, inevitably leads to destruction and modification of surface molecules of the substrate.

### Film Composite and its Manufacture According to the First Embodiment

The production of microfluidic structures according to the first embodiment is described in DE 10 2020 114 621 A1.

The method for producing a film composite according to the first embodiment comprises the following steps:
(a) applying a layer of a curable embossing lacquer to a first surface side of a carrier film to form an embossing lacquer layer in an embossing lacquer section,
(b) applying a layer of a curable bonding lacquer to the first surface side of the carrier film to form a bonding lacquer layer in a bonding lacquer section,
(c) embossing recessions in the embossing lacquer layer obtained in step (a) with an embossing tool,
(d) partial curing of the embossing lacquer layer embossed in step (c),
(e) removing the embossing tool after step (d),
(f) forming through-holes through the structure obtained in step (e) from the first surface side to the second surface side of the carrier film and folding the structure between the embossing lacquer section and the bonding lacquer section to form two film layers,
(g) superimposing the two film layers obtained in step (f) to form a composite so that the recessions of the embossing lacquer layer of the embossing lacquer section of one film layer are sealed by the bonding lacquer layer of the bonding lacquer section of the other film layer to form microfluidic structures,
(h) curing the partially cured embossing lacquer layer and the bonding lacquer layer of the composite obtained in step (g) to form covalent bonds between these layers, thereby obtaining the film composite.

Preferably, the curable bonding lacquer is a curable embossing lacquer which is embossed and partially cured according to steps (c) to (e).

A preferred film composite according to the invention can be produced according to the method of the first embodiment.

In this embodiment, the microfluidic structures are tightly sealed. The term “tightly” means here that the covering of a microfluidic structure cannot be detached non-destructively, for example by heating. The basis for the tight cover in the present invention is the covalent bond between the initial structures. The tight closure of the microfluidic structures is achieved by the surfaces of the elements being chemically bonded to each other, i.e. covalently bonded. This creates a layered structure with an embedded microfluidic structure that can no longer be separated into its individual parts. A hermetic seal also prevents the penetration of foreign substances and impurities that could falsify the analysis result and also allows temperature-controlled processes to run (e.g. on-chip amplification).

The microfluidic structures are sealed exclusively with hardened embossing lacquer and hardened bonding lacquer. This means that the base, the side walls and the cover consist exclusively of these hardened lacquers, with the exception that when embossing a depth of 100%, the base is the carrier film. This embossing excludes other substances, for example an adhesive between the bottom part and the cover part.

The curable embossing lacquer and the curable bonding lacquer may be the same or different from each other. The terms “lacquer” and “lacquer layer” used in this embodiment refer both to the curable embossing lacquer and, independently thereof, to the curable bonding lacquer or the layers formed therefrom, unless otherwise indicated. The lacquer layer comprises or consists of a lacquer.

The lacquer can be cured, for example thermally or by using UV rays or electron beams. Curing with UV rays is preferred. The lacquer preferably contains at least one compound with polymerisable C—C double bonds. The compound may be selected from the group consisting of acrylates, methacrylates, vinyl ethers, allyl ethers, propenyl ethers, alkenes, dienes, unsaturated esters—in particular polyitaconates, allyl triazines, allyl isocyanates and N-vinyl amides. (Meth)acrylates are preferred. Urethane acrylates are preferred. In addition to the compound with polymerisable C—C double bonds, the coating may also contain thiol-containing compounds. The polyitaconates are a special case of the unsaturated esters—but still very much worth mentioning.

Suitable UV-curable lacqueres are based, for example, on polyethylene glycol diacrylates (PEGDA), possibly with 1-10 mass percent of higher-functional acrylates, or lacquer systems based on highly crosslinking multifunctional polyether, polyester or polyurethane acrylate systems. Preferred are polyethylene glycol diacrylates, ethoxylated (very important!) trimethylolpropane triacrylates, acryloyl morpholine, ethoxylated pentaerythritol tetraacrylate, hexanediol diacrylate and trifunctional urethane acrylate oligomer. Other examples include triglycerol diacrylate/glycerol 1,3-diglycerolate diacrylates and polyethoxylated pentaerythritol diacrylate and triacrylate. The non-acrylated of the four OH groups of pentaerythritol can interact with the aqueous solutions in the microfluidic structure or be used for functionalisations.

UV-curable acrylic lacqueres are particularly preferred. They are easy to process in a roll-to-roll process because they do not require any pre-processes (prebake such as SU8) and crosslink very quickly (in less than 1 s). They are also very adjustable with regard to their surface energy and thus their wetting properties, which play an essential role in capillary-driven microfluidics.

The lacquer may contain 0.05 to 5 mass percent of photoinitiators that cause crosslinking under UV radiation, for example photoinitiators based on acyl phosphine oxides, or oligomeric polyfunctional alpha hydroxy ketones or monomeric alpha hydroxy ketones.

The embossing lacquer and the bonding lacquer can contain additives independently of each other. Examples of such additives are surface-active anti-adhesive additives and reactive thinners.

In order to reduce adhesion of the embossing lacquer to the embossing tool and to facilitate removal of the embossing tool after embossing, a surface-active anti-adhesion additive may be added to the embossing lacquer used as starting material, which may be silicone-containing or fluorine-containing. In particular, the additive is at least one member selected from the group comprising silicone-containing or fluorine-containing additives. These additives help to reduce adhesion and facilitate detachment of the embossing lacquer from the embossing tool. Said anti-adhesion additives may be at least partially present in the embossed structural layer after carrying out the process according to the invention, for example in an amount of 0.1 to 3% by weight.

A reactive thinner may be included in the embossing lacquer and/or the bonding lacquer. A reactive diluent contains low-molecular compounds to adjust the viscosity of the lacquer.

The lacquer layer material is applied to a substrate film. As the lacquer layer material is not cured, i.e. not polymerised, it has a low viscosity so that it can be applied, for example, by brushing or by casting. Preferably, it is applied by engraving or slot die coating.

The viscosity is adjusted so that the material can be applied easily. On the other hand, it must be viscous enough to preserve the structure formed during embossing. To meet both requirements, the material applied to the substrate can first be moderately irradiated so that partial curing and thus an increase in viscosity takes place. The lacquer may already be solid after partial curing, but it will have non-reacted reactive groups. The desired low viscosity of the lacquer layer material is preferably achieved at room temperature. This allows the application of the lacquer layer material at low temperatures, such as room temperature.

The embossed lacquer layer has a thickness, i.e. an extent perpendicular to the contact surface with the substrate film, of preferably 100 nm to 1000 μm, more preferably 1 μm to 100 μm. In individual embodiments, the thickness may be 1 μm to 50 μm.

The bonding lacquer layer is preferably thinner than the embossing lacquer layer and preferably has a thickness of less than 5 μm, more preferably less than 1 μm. A preferred combination is an embossing lacquer layer with a thickness of 1 μm to 100 μm and a bonding lacquer layer with a thickness of less than 1 μm.

The steps of the method of the first embodiment are described in detail below.

### Steps (a) and (b):

The application of a layer of a curable embossing lacquer and a layer of a curable bonding lacquer to the same surface side of the carrier film is carried out, for example, with a roll-to-roll process, in which the application of the lacquer layer to the substrate film is carried out, for example, by means of a slit nozzle or by gravure printing with an engraving roller. When folded according to the fanfold principle, the embossing lacquer layer and the bonding lacquer layer can be formed in different sections of the same surface side of the substrate film so that they can later be placed on top of each other by folding them over at a folding edge located between the two sections. When folded according to a different principle, for example by folding two outer film layers onto a middle film layer, the embossing lacquer layer and the bonding lacquer layer can be formed on opposite surface sides, i.e. front side and rear side, of the carrier film.

The thickness of the bonding lacquer layer is preferably small to allow faster curing in step (h) and to prevent blocking of the recesses when forming the bond in step (g). If the bonding lacquer layer is too thick, in step (g) the still liquid material can flow or be pressed into the recesses of the embossing lacquer layer even before the surfaces and layers are chemically cured, i.e. covalently bonded. For example, the bonding lacquer layer has a thickness of less than 1 μm. A thickness of the bonding lacquer layer in the nanometre range also enables an increase in the number of film layers for a given height of the film composite.

Since the embossing lacquer for the application in step (a) is preferably low viscosity and in a preferred embodiment the composition of the embossing lacquer and the bonding lacquer is the same, the bonding lacquer is also low viscosity when applied. In this case, it may be preferred or necessary that the carrier film with the bonding lacquer is arranged in such a way that the bonding lacquer layer lies on top of the carrier film under the effect of gravity, since otherwise it cannot be guaranteed that the thin liquid bonding lacquer layer remains on the carrier film until the laminating step (g). In the first embodiment, the embossing lacquer and the bonding lacquer are applied to the same surface side of the carrier film. This surface side is preferably the upper side of the carrier film, so that the mentioned problems are avoided.

Alternatively, however, the adhesion of the bonding lacquer to the carrier film occurs independently of the effect of gravity and is based on non-covalent adhesion. This adhesion is achieved by adjusting the surface energies of the cover film and the bonding lacquer, by selecting a suitable bonding lacquer material and/or in particular by using a thin layer of the bonding lacquer.

To make the bonding lacquer layer more viscous and, if necessary, to bind it covalently to the cover film, it can be partially cured. However, this leads to a lower adhesion after curing in step (h).

In one embodiment of the first embodiment, the carrier film may have an embossed lacquer layer on both surface sides, each of the two embossed lacquer layers having its embossing on the surface side facing away from the carrier, so that a structure embossed on both sides can be obtained. This can be done, for example, by a double pass in a roll-to-roll process.

### Step (c):

The embossing of recessions in the embossing lacquer layer obtained in step (a) with an embossing tool is carried out in at least a partial area of the embossing lacquer layer and comprises forming a recession in the plane of the embossing lacquer layer.

Embossing is carried out with an embossing tool, e.g. a punch, and can be implemented in a continuous roll-to-roll process if a cylindrical punch is used.

UV-NIL can be used, in which a nanostructured stamp with the inverted profile of the desired structure is used as a negative mould and pressed into the embossing lacquer, in which the desired structure is then embossed as a positive mould. A preferred method according to the invention is a high-throughput production process (roll-to-roll UV-NIL) for microfluidic structures, in which the channels and chambers are produced on large polymer substrates in roll format.

The depth of the embossing, i.e. the extent of the recession in the z-direction, can be 1 to less than 100% of the thickness of the embossing lacquer layer. Preferably it is 50 to less than 100%, more preferably 80 to 100% of the thickness of the embossing lacquer layer.

The bonding lacquer layer can also be embossed. In this way, the embossing of the embossing lacquer layer on the carrier film and the embossing of the bonding lacquer layer can complement each other structurally and functionally, and a wide variety of structures with microfluidic structures and cavities can be obtained that are not obtainable if only the embossing lacquer layer is embossed. The cavities of the bonding lacquer layer can be arranged above the cavities of the embossing lacquer layer, so that a higher cavity is created. In this way, microfluidic structures with a large volume can be formed, which can be used, for example, as storage chambers or reaction chambers.

### Step (d):

The extent of partial curing of the embossing lacquer layer embossed in step (c) is adjusted so that, on the one hand, a sufficient amount of reactive groups remains for subsequent curing and bonding with the bonding lacquer and, on the other hand, sufficient partial curing takes place to ensure the required stability of the structures for demoulding and folding. For this purpose, the following parameters are optimised: Polymerisation speed of the stamping lacquer (especially the type and content of the photoinitiator), UV intensity, web speed, transmission of the substrate film.

### Step (e):

After embossing and partial curing, the embossing tool is removed. To facilitate this step and to increase the quality of the embossing, surface-active non-stick additives can be added to the embossing lacquer used in step (a).

### Step (f):

The through-holes are created in the z-direction by the structure obtained in step (e). For example, the formation of through-holes takes place at locations where embossed recessions are present. Depending on whether the carrier film is covered with embossing lacquer on one side or both sides at the respective location and whether the embossing has a depth of 100% or less, the material of the carrier film and possibly material of the embossing lacquer must be removed. The through-holes can be created with the help of a laser.

The structure obtained in step (e) is also folded before or after the formation of the through-holes. The folding is preferably done at folding edges, which are created, for example, via laser structuring. The scoring of folding edges enables the self-alignment of the film layers.

### Step (g)

The two film layers are placed on top of each other. Since it is preferred that the two film layers are congruent, the two film layers can be placed on top of each other with an exact fit. If necessary, the two layers of film can be pressed together under low pressure and held in this state.

The embossing lacquer layer of one film layer is covered directly and over its entire surface by the bonding lacquer layer of the other film layer, with the exception of the recessions in the embossing. Preferably, there is neither embossing lacquer nor bonding lacquer in the recessions of the embossing, so that cavities are created. The two film layers form a sandwich-like composite with outer carrier films and inner lacquer layers that are bonded together and have microfluidic structures.

### Step (h):

In the film composite according to the invention, the embossing lacquer layer and the bonding lacquer layer are completely cured, i.e. polymerised and crosslinked. This complete curing takes place in step (h). The curing is preferably a photopolymerisation and is carried out by exposure to a radiation. This radiation is preferably light of suitable wavelength, for example UV light. The dose of radiation is high enough to ensure complete polymerisation of the starting material. Preferably, an overdose is used to ensure the completeness of the curing.

The partial curing in step (d) and the curing in step (h) may be carried out in the same or different ways, the same way being preferred. The partial curing and the hardening can be carried out independently of each other thermally or by using UV rays or electron beams. Preferably, the partial curing in step (d) and the curing in step (h) are performed by UV irradiation.

### Film Composite and its Manufacture According to the Second Embodiment

The fabrication of microfluidic structures according to the second embodiment is described in WO 2020/187990 A1.

The method for producing a film composite according to the invention according to the second embodiment comprises the following steps:
(a) applying a layer of an embossing lacquer containing monomers radically polymerisable to a polymer to a first surface side of a carrier film to form an embossing lacquer layer in an embossing lacquer section,
(b) applying a cover layer to the first surface side of the carrier film to form a cover layer in a cover layer section,
(c) embossing recessions in the embossing lacquer layer obtained in step (a) with an embossing tool,
(d) polymerising the monomers contained in the embossing lacquer layer into a polymer to form a thermoplastic embossing lacquer layer on the carrier film,
(e) removing the embossing tool after step (d),
(f) forming through-holes through the structure obtained in step (e) from the first surface side to the second surface side of the carrier film and folding the structure between the embossing lacquer section and the cover layer section to form two film layers,
(g) thermally bonding the two film layers obtained in step (f) to each other so that the recessions of the embossing lacquer layer of the embossing lacquer section of one film layer are sealed by the cover layer of the cover layer section of the other film layer, forming microfluidic structures, whereby the film composite is obtained.

Preferably, the cover layer is a thermoplastic lacquer layer which is embossed and polymerised according to steps (c) to (e).

In the film composite according to the invention, microfluidic structures are formed in that film layers with embossed recessions are covered by the respective adjacent film layer. In the description of the second embodiment, the adjacent film layer or its lowest layer is referred to as the cover layer for illustrative purposes. However, this cover layer is not necessarily located above the film layer with the embossed recessions. Rather, it can also be located under the film layer in an analogous manner if the embossing lacquer layer is arranged on the underside of the carrier film. A cover layer can also be arranged on both sides of the film layer.

The application of the embossing lacquer layer and the cover layer in steps (a) and (b) is basically carried out as in the first embodiment, but the materials differ. The embossing in steps (c) to (e) is also performed as described in the first embodiment, but in step (d) the partial curing of the first embodiment and the polymerisation of the second embodiment differ. Step (f) is the same in both embodiments.

The method of the second embodiment differs from the method of the first embodiment essentially in the nature of the starting materials of the embossing lacquer layer and the bonding layer or cover layer.

The embossed lacquer layer used in the second embodiment has the features that it is a lacquer layer which is thermoplastic, embossed and radically polymerised.

In one embodiment of the second embodiment, the carrier film may have an embossed lacquer layer on both surface sides, each of the two embossed lacquer layers having its embossing on the surface side facing away from the carrier, so that a structure embossed on both sides can be obtained. This can be done, for example, by a double pass in a roll-to-roll process.

The base material for the coating layer is not or not completely polymerised and is transferred into the coating by radical polymerisation, preferably photopolymerisation or thermal polymerisation. It is therefore monomers or oligomers. A prepolymer and a chain extender can also be used.

The base material is applied to a carrier layer, e.g. a polymer substrate. Because the base material is not cured, i.e. not polymerised, it has a desired viscosity so that it can be applied, for example, by brushing. Preferably, the material is liquid and can thus be applied by pouring. In the roll-to-roll process, the lacquer is applied to the substrate by means of a slit nozzle, for example, or by gravure printing with an engraving roller.

The viscosity is adjusted so that the material can be applied easily. On the other hand, it must be viscous enough to preserve the structure formed during embossing.

A coating layer is used that forms only or essentially only linear polymer chains during polymerisation, e.g. when irradiated with UV light. These chains do not or hardly cross-link with each other, so that no or only slight cross-linking occurs. Such a coating layer shows thermoplastic behaviour and is thus thermally deformable.

The base material for the coating layer is completely polymerised in step (d). The polymerisation is preferably a photopolymerisation and takes place by exposure to radiation. This radiation is preferably light of suitable wavelength, for example UV light. The dose of radiation is high enough to ensure complete polymerisation of the starting material. Preferably, an overdose is used to ensure the completeness of the polymerisation.

In the film composite according to the second embodiment, the cover layer corresponds to the adjacent film layer or its lowermost layer. The cover layer material can therefore be either the carrier film of the adjacent film layer or a layer applied thereto, for example an embossed lacquer layer. The material of the cover layer is not restricted and is preferably also thermoplastic.

After folding the embossed surface side, the cover layer faces the embossing lacquer layer, so that due to the recessions in the embossing lacquer layer, there are cavities between the two layers that form microfluidic structures.

The cover layer can have an embossing. In this way, the embossing of the embossing lacquer layer on the carrier film and the embossing of the cover layer can complement each other structurally and functionally, and a wide variety of structures with microfluidic structures and cavities can be obtained that are not obtainable if only the embossing lacquer layer is embossed. The recessions of the cover layer can be arranged above the recessions of the embossing lacquer layer, so that a higher cavity is created.

In step (g), thermal bonding is performed to bond the embossing lacquer layer to the cover layer. The term “thermal bonding” as used herein means creating a bond between two components, e.g. two layers, by a thermoplastic welding type process which involves heating at least one of the two components. Examples of thermal bonding are hot pressing, hot lamination, thermocompression bonding and ultrasonic and laser welding. Thermal bonding thus means, on the one hand, that the bond between two components can be created by heating at least one of the two components, contacting the two components and then cooling to form the composite material from the two components, and, on the other hand, that this bond can also be released again by heating in a non-destructive manner.

Thermal bonding to join the embossing lacquer layer to the cover layer is carried out at a temperature (bonding temperature) at which the materials of the two layers are softened to such an extent that a firm bond is formed between the layers after cooling. The bonding temperature is selected depending on the materials of the layers in such a way that, on the one hand, the desired bond between the layers is formed and, on the other hand, the embossing in the embossing lacquer layer is not damaged or adversely changed by the softening. Therefore, the softening temperatures of the layers should preferably be similar. The easiest way to achieve this is to use the same materials for the embossing lacquer layer and the cover layer with consequently the same softening properties.

In one embodiment, the embossing lacquer layer may have a slightly higher softening temperature than the cover layer. This can help to ensure that the embossing is not damaged or altered during softening and bonding.

If the embossing lacquer layer is bonded to the cover layer exclusively by thermal bonding, no covalent bonds are created between the two layers, so that the two layers could also be separated again by heating.

### Application Example

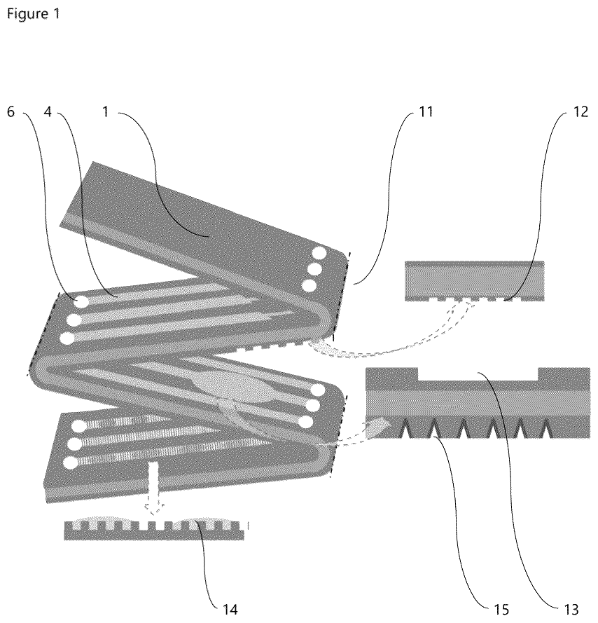

An example of an application of the film composite according to the invention is DNA amplification by means of isothermal amplification (LAMP), illustrated in FIG. 1, which reduces the effort of sample preparation and thus saves time and costs. In addition, the complexity of the test procedure is reduced, which facilitates its widespread use as a rapid test in medical practices and in the private environment.

The heating agent required for isothermal amplification can be filled directly into the recessions created after embossing. For example, a metal ink, e.g. Ag nanoparticle ink, can be filled in as the heating agent by means of capillary action. In this way, microfluidically filled electrodes can be created, which can be used as film heaters for isothermal DNA amplification. Depending on the requirements, the heating channels can be embossed on the same surface side of the film as the microfluidic structures or on the opposite surface side. This significantly reduces the number of process steps and thus the costs compared to the production of a separate heating film. At the same time, the performance is increased, as the heat transfer is more direct without intermediate adhesive layers and additional substrates, and thus less energy is consumed.

The fabrication of the hierarchical structures (channels with micro- or nanoscale superstructure) can take place in a single embossing step. For example, the overstructure is fabricated as part of the master fabrication, either by a common photolithographic step or by preceding embossing of the micro- or nanostructure, followed by photolithography over the embossed structure. For example, hierarchical structures in the measurement channel allow the sensor surface area to be increased and the sensitivity and selectivity to be increased.

### LIST OF REFERENCE SIGNS

1 Film layer
2 Carrier film
3 Embossing lacquer layer
4 Recess
5 Microfluidic structure
6 Through-hole
7, 9 Recess or microfluidic structure, respectively, with heating medium
8, 10 Recess or microfluidic structure, respectively, with nanostructures
11 Folding edge
12 Hydrophobic valve
13 Chamber for isothermal PCR
14 Microstructured surface
15 Microchamber with heating medium
16 Reaction chamber
17 Connecting recess

What is claimed is:

1. A film composite which is folded from a single film to form a plurality of layered film layers (1) and comprises microfluidic structures (5) in different planes, wherein the microfluidic structures are formed in that film layers with embossed recesses are covered by the respective film layer adjacent in the layering direction, and wherein microfluidic structures of one plane are connected to microfluidic structures of another plane via through-holes in at least one film layer.

2. The film composite according to claim 1, which is folded in a fan-shaped manner or is obtainable by folding a film, which has two folding edges and thus a central film layer and a first and a second outer film layer, in such a way that the first outer film layer is folded over the central film layer and the second outer film layer is subsequently folded over the first film layer.

3. The film composite according to claim 1, wherein at least one of the film layers comprises a carrier film and, on one surface side of the carrier film, a lacquer layer with embossed recesses; and/or wherein at least one of the film layers comprises a carrier film and, on both surface sides of the carrier film, a lacquer layer with embossed recesses on at least one of the two surface sides of the carrier film.

4. The film composite according to claim 1, wherein the film is folded at folding edges created by laser cutting of the films and/or wherein the through-holes are created by laser cutting.

5. The film composite according to claim 1, which consists of congruent film layers, which lie directly on top of each other over their entire surface, and folded edges.

6. The film composite according to claim 1, wherein microfluidic structures communicating with one another are formed in different planes, in that two film layers adjacent in the layering direction have embossed recessions which face one another and only partially overlap one another in the layering direction.

7. The film composite according to claim 1, comprising in the microfluidic structures at least one modification selected from functionalisation, nanostructuring, hydrophobisation and heating agent filling.

8. The film composite according to claim 1, wherein at least one of the film layers has a carrier film and, on at least one surface side of the carrier film, a lacquer layer with embossed recessions, wherein either the recessions are present in a lacquer layer of the film layers and the lacquer layer is bonded to the adjacent film layer by UV curing, or the recessions are present in a thermoplastic lacquer layer of the film layers and the lacquer layer is bonded to the adjacent film layer by exclusively thermal bonding.

9. The film composite according to claim 1, which is folded in a fan-shaped manner and consists of congruent film layers, which lie directly over one another over their entire surface, and folding edges, wherein the folding edges and the through-holes are produced using a laser, and wherein the film layers have a carrier film and a lacquer layer with recessions and the lacquer layer is bonded to the adjacent film layer by UV curing.

10. A method of manufacturing a film composite according to claim 1, comprising the steps of:
(a) applying a layer of a curable embossing lacquer to a first surface side of a carrier film to form an embossing lacquer layer in an embossing lacquer section,
(b) applying a layer of a curable bonding lacquer to the first surface side of the carrier film to form a bonding lacquer layer in a bonding lacquer section,
(c) embossing recessions in the embossing lacquer layer obtained in step (a) with an embossing tool,
(d) partial curing of the embossing lacquer layer embossed in step (c),
(e) removing the embossing tool after step (d),
(f) forming through-holes through the structure obtained in step (e) from the first surface side to the second surface side of the carrier film, and folding the structure between the embossing lacquer section and the bonding lacquer section to form two film layers,
(g) superimposing the two film layers obtained in step (f) to form a composite, so that the recessions of the embossing lacquer layer of the embossing lacquer section of one film layer are sealed by the bonding lacquer layer of the bonding lacquer section of the other film layer to form microfluidic structures, and
(h) curing the partially cured embossing lacquer layer and the bonding lacquer layer of the composite obtained in step (g) to form covalent bonds between these layers, thereby obtaining the film composite.

11. The method according to claim 10, wherein the curable bonding lacquer is a curable embossing lacquer which is embossed and partially cured according to steps (c) to (e).

12. The method of manufacturing a film composite according to claim 1, comprising the steps of:
(a) applying a layer of an embossing lacquer containing monomers radically polymerisable to a polymer to a first surface side of a carrier film to form an embossing lacquer layer in an embossing lacquer section,
(b) applying a cover layer to the first surface side of the carrier film to form a cover layer in a cover layer section,
(c) embossing recessions in the embossing lacquer layer obtained in step (a) with an embossing tool,
(d) polymerising the monomers contained in the embossing lacquer layer into a polymer to form a thermoplastic embossing lacquer layer on the carrier film,
(e) removing the embossing tool after step (d),
(f) forming through-holes through the structure obtained in step (e) from the first surface side to the second surface side of the carrier film, and folding the structure between the embossing lacquer section and the cover layer section to form two film layers, and
(g) thermally bonding the two film layers obtained in step (f) to each other, so that the recessions of the embossing lacquer layer of the embossing lacquer section of one film layer are sealed by the cover layer of the cover layer section of the other film layer to form microfluidic structures, whereby the film composite is obtained.

13. The method according to claim 12, wherein the cover layer is a thermoplastic embossed lacquer layer which is embossed and polymerised according to steps (c) to (e).

14. The method according to claim 10, wherein the embossing is carried out in a roll-to-roll process.

15. The use of a film composite according to claim 1 for the amplification of DNA by polymerase chain reaction or isothermal amplification.
